(19) United States
(12) Patent Application Publication
(10) Pub. No.: US 2008/0020057 A1
(43) Pub. Date: Jan. 24, 2008

(54) Personal care composition containing microemulsified wax particles

(76) Inventors: Michael Frederick Niebauer, Cincinnati, OH (US); Brandon Scott Lane, Hamilton, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/488,257

(22) Filed: Jul. 18, 2006

(51) Int. Cl. A61K 9/50; A61K 31/415; A61Q 5/12; A61K 31/695

(52) U.S. Cl. 424/502; 514/399; 514/63

(57) ABSTRACT

Personal care compositions comprise (a) from about 5 wt. % to about 50 wt. % of a detersive surfactant, (b) from about 0.05 wt. % to about 20 wt. % of wax particles having a melting point of at least about 100° C. and an average mean particle size as measured in said personal care composition of less than about 0.15 μm; and (c) at least about 20 wt. % of a cosmetically acceptable medium; wherein the personal care composition is substantially clear.

### FIELD OF THE INVENTION

The present invention relates to personal care compositions containing particles. More specifically, it relates to hair care compositions containing microemulsified wax particles which provide volume and conditioning and allow for the composition to be substantially clear.

### BACKGROUND OF THE INVENTION

Solid particles are known for use as benefit agents in a variety of personal care compositions. Solid particles can impart benefits both to the compositions comprising them or surfaces to which the compositions are applied. For example, solid particles can be used as pigments or coloring agents, opacifiers, pearlescent agents, feel modifiers, oil absorbers, skin protectants, matting agents, friction enhancers, slip agents, conditioning agents, exfoliants, odor absorbers, or cleaning enhancers. Additionally, many active ingredients useful as treatment agents for various disorders or socially embarrassing conditions are available and typically used in solid particulate form including antiperspirant agents, anti-dandruff agents, antimicrobials, antibiotics, and sunscreens.

Typically when it is desired to modify the properties of a surface through application of particles, the particles are applied via leave-on preparations that are rubbed, sprayed, or otherwise applied directly onto the surface to be affected. Typical personal care preparations suitable for delivery of solid particles to hair or skin surfaces include moisturizers, lotions, creams, loose or pressed powders, sticks, tonics, gels, and various sprays such as aerosol or pump sprays. These products are typically applied directly to the surface whereupon particles are deposited and retained by the composition itself or by residual non-volatile elements of the composition after evaporation and drying.

It is known to formulate solid particle benefit agents into rinse-off or cleansing compositions such as hair rinses, shampoos, liquid and bar soaps, conditioners, or colorants. Frequently, the solid particle benefit agent is used to affect the overall appearance, stability or aesthetics of the composition itself. For example, it is known to add colorant particles, pigments, or pearlescent agents to compositions to improve the acceptability and attractiveness of the product to potential consumers. It is also known to add particulate benefit agents to affect the in use performance, appearance, or aesthetic properties of the composition or to provide a tactile signal to the user. For example, exfoliant particles are frequently used in cleansing compositions to improve abrasion and removal of oils and dirt from washed surfaces and to impart a perceptible “scrubbing” sensation to the user. Typically, such solid particle agents are not intended or desired to be deposited onto the substrate and are removed during dilution and rinsing of the composition from the surface to which they are applied.

It is also known to formulate solid particles into rinse-off or cleansing compositions to improve the volume, body, or fullness of the hair through increased interactions between hair fibers. However, compositions containing solid particles intended to provide increased interaction between hair fibers frequently result in negative conditioning, wet detangling, and wet combing performance due to the frictional impact of the particles. Therefore, the negative conditioning and combing effects associated with the deposition of particles often outweigh the positive hair volume and body results. Prior attempts to mitigate the negative conditioning attributes of particle containing compositions have either been insufficient or have negatively impacted the deposition or benefit associated with the particle.

Substantially clear personal cleansing compositions provide the ability to enhance product packaging by allowing the consumer to view both the internal and external surfaces of the package. However, previous attempts to formulate substantially clear personal care compositions, containing particles, have lacked prolonged visual clarity and/or provide insufficient volume. The Brownian motion of charged particles causes particles to collide with each other, and each collision tends to result in adhesion of the particles (aggregation). Neutrally charged particles have a reduced tendency to aggregate because they do not exhibit the same inter-particle forces observed by charged particles. However, neutral particles formulated in the personal care compositions tend to provide insufficient friction to provide significant volume to hair.

Accordingly, a need still exists for a physically stable, substantially clear rinse-off personal care composition which is capable of effectively depositing and retaining particle benefit agents on the treated surface to deliver improved hair volume, body, and fullness, while simultaneously delivering improved conditioning and combing performance.

### SUMMARY OF THE INVENTION

The present invention is directed to a personal care composition comprising:
a) from about 5 wt. % to about 50 wt. % of a detersive surfactant,
b) from about 0.05 wt. % to about 20 wt. % of wax particles having a melting point of at least about 100° C. and an average mean particle size as measured in said personal care composition of less than about 0.15 μm,
c) at least about 20 wt. % of a cosmetically acceptable medium; and
wherein said personal care composition is substantially clear.

These and other features, aspects, and advantages of the present invention will become evident to those skilled in the art from a reading of the present disclosure.

### DETAILED DESCRIPTION

While the specification concludes with claims that particularly point out and distinctly claim the invention, it is believed the present invention will be better understood from the following description.

The personal care composition of the present invention comprises a detersive surfactant, wax particles, and a cosmetically acceptable medium wherein the composition is substantially clear. Each of these essential components, as well as preferred or optional components, is described in detail hereinafter.

All percentages, parts, and ratios are based upon the total weight of the compositions, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

Herein, “comprising” means that other steps and other ingredients which do not affect the end result can be added. This term encompasses the terms “consisting of” and “consisting essentially of.” The compositions and methods/processes can comprise, consist of, and consist essentially of the essential elements and limitations of the invention described herein, as well as any of the additional or optional ingredients, components, steps, or limitations described herein.

The term “charge density,” as used herein, refers to the ratio of the number of positive charges on a monomeric unit of which a polymer is comprised to the molecular weight of said monomeric unit. The charge density multiplied by the polymer molecular weight determines the number of positively charged sites on a given polymer chain.

The term “load-sensitive deposit,” as used herein, means a material which, at very low force applied to the hair (e.g., the typical force present with hair-to-hair contact), acts to increase hair friction relative to the hair friction of clean hair, and, at higher force applied to the hair (e.g., the force present during combing), acts to decrease hair friction relative to the hair friction at very low force.

The term “physically stable,” as used herein, means the particles do not undergo a physical transformation, altering the external appearance of the personal care composition for at least six (6) months, and more preferably at least one (1) year from the date of formulation and under ambient conditions.

The term “ambient conditions,” as used herein, unless otherwise specified, refers to surrounding conditions at one (1) atmosphere of pressure, 50% relative humidity, and 25° C.

The term “substantially clear,” as used herein, means the personal care composition has a percent transmittance of at least about 70%.

The term “polymer,” as used herein, includes materials whether made by polymerization of one type of monomer or made by two (i.e., copolymers) or more types of monomers.

The term “water-soluble,” as used herein, means that a substance is soluble in water in the present composition. In general, the substance should be soluble at 25° C. at a concentration of at least about 0.1% by weight of the water solvent, preferably at least about 1%, more preferably at least about 5%, even more preferably at least about 15%.

Viscosity measurements are achieved using a Brookfield R/S Rheometer at a shear rate of 2 s⁻¹ for 3 minutes.

### Detersive Surfactant

The present compositions comprise a detersive surfactant. The detersive surfactant component is included to provide cleaning performance to the composition. The detersive surfactant component comprises anionic detersive surfactant, zwitterionic or amphoteric detersive surfactant, or a combination thereof. Such surfactants should be physically and chemically compatible with the essential components described herein, or should not otherwise unduly impair product stability, aesthetics, or performance.

Suitable anionic detersive surfactant components for use in the composition herein include those which are known for use in hair care or other personal care cleansing compositions. The concentration of the anionic surfactant component in the composition should be sufficient to provide the desired cleaning and lather performance, and generally range from about 5% to about 50%, preferably from about 8% to about 30%, more preferably from about 10% to about 25%, even more preferably from about 12% to about 22%.

Preferred anionic surfactants suitable for use in the compositions are the alkyl and alkyl ether sulfates. These materials have the respective formulae ROSO₃M and RO(C₂H₄₀)ₓSO₃M, wherein R is alkyl or alkenyl of from about 8 to about 18 carbon atoms, x is an integer having a value of from 1 to 10, and M is a cation such as ammonium, alkanolamines, such as triethanolamine, monovalent metals, such as sodium and potassium, and polyvalent metal cations, such as magnesium, and calcium.

Other suitable anionic detersive surfactants are the water-soluble salts of organic, sulfuric acid reaction products conforming to the formula R¹—SO₃-M wherein R¹ is a straight or branched chain, saturated, aliphatic hydrocarbon radical having from about 8 to about 24, preferably from about 10 to about 18, carbon atoms; and M is a cation described hereinbefore.

Still other suitable anionic detersive surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278.

Other anionic detersive surfactants suitable for use in the compositions are the succinnates, examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic detersive surfactants include olefin sulfonates having from about 10 to about 24 carbon atoms. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non-limiting example of such an alpha-olefin sulfonate mixture is described in U.S. Pat. No. 3,332,880.

Another class of anionic detersive surfactants suitable for use in the compositions is the beta-alkyloxy alkane sulfonates. These surfactants conform to the formula:

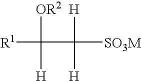

where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore.

Preferred anionic detersive surfactants for use in the compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate, and combinations thereof.

Suitable zwitterionic or amphoteric detersive surfactants for use in the composition herein include those which are known for use in hair care or other personal care compositions. Concentration of such amphoteric detersive surfactants preferably ranges from about 0.5% to about 20%, preferably from about 1% to about 10%. Non-limiting examples of suitable zwitterionic or amphoteric surfactants are described in U.S. Pat. Nos. 5,104,646 and 5,106,609, both to Bolich Jr. et al.

Amphoteric detersive surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Preferred amphoteric detersive surfactants for use in the present invention include cocoamphoacetate, cocoamphodiacetate, lauroamphoacetate, lauroamphodiacetate, and mixtures thereof.

Zwitterionic detersive surfactants suitable for use in the composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate, or phosphonate. Zwitterionics such as betaines are preferred.

The present compositions may further comprise additional surfactants for use in combination with the anionic detersive surfactant component described hereinbefore. Suitable optional surfactants include nonionic and cationic surfactants. Any such surfactant known in the art for use in hair or personal care products may be used, provided that the optional additional surfactant is also chemically and physically compatible with the essential components of the composition, or does not otherwise unduly impair product performance, aesthetics, or stability. The concentration of the optional additional surfactants in the composition may vary with the cleansing or lather performance desired, the optional surfactant selected, the desired product concentration, the presence of other components in the composition, and other factors well known in the art.

Non-limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678; 2,658,072; 2,438,091; and 2,528,378.

### Wax Particles

The present compositions comprise wax particles. The wax particles have an average mean particle size of less than about 0.15 μm, preferably from 0.001 μm to about 0.15 μm, more preferably from about 0.001 μm to about 0.10 μm. These particles are physically stable and may be formulated in substantially clear compositions. The particle size of the wax particles is important to formulating substantially clear compositions. Personal care compositions become increasingly clear as particle size decreases. Accordingly, particle sizes of less than about 0.15 μm are useful for formulating substantially clear compositions.

Typically, personal care compositions containing small charged particles do not remain clear for prolonged periods of time. The Brownian motion of charged particles causes particles to collide with each other, and each collision tends to result in adhesion of the particles (aggregation). Addition of salts and surfactants tends to increase inter-particle collisions, resulting in increased agglomeration. It has been surprisingly discovered that the wax particles of the present invention do not suffer from agglomeration in the presence or absence of surfactants or salts. It is believed that because wax particles are neutrally charged, the particles demonstrate less interaction, resulting in physically stable compositions. Although neutrally charged particles generally do not provide sufficient friction to provide significant hair care benefits, the present compositions comprise wax particles which have a sufficiently high melting temperature to provide improved friction for increased hair volume as discussed below. Thus, the present particles may be incorporated into personal care compositions while maintaining substantial clarity.

The wax particles have a melting point of at least about 100° C. Preferably, the wax particles have a melting point of from about 105° C. to about 200° C., more preferably from about 110° C. to about 170° C., and even more preferably from about 115° C. to about 160° C. As used herein, the melting point refers to the temperature at which the particles transition to a liquid or fluid state. Without being bound by theory, it is believed that friction increases with increasing melting point temperature. Therefore, wax particles having higher melting temperatures provide increased friction, which results in an increased volume benefit on human hair.

The transparency of the composition is measured using Ultra-Violet/Visible (UV/VIS) Spectrophotometry, which determines the absorption or transmission of UV/VIS light by a sample. A light wavelength of 600 nm is adequate for characterizing the degree of clarity of cosmetic compositions. Typically, it is best to follow the specific instructions relating the specific spectrophotometer being used. In general, the procedure for measuring percent transmittance starts by setting the spectrophotometer to the 600 nm. Then a calibration “blank” is run to calibrate the readout to 100 percent transmittance. The test sample is then placed in a cuvette designed to fit the specific spectrophotomer and the percent transmittance is measured by the spectrophotomer at 600 nm.

The present compositions comprise wax particles from about 0.05% to about 20%, preferably from about 0.1% to about 10%, more preferably from about 0.1% to about 5%, even more preferably about 0.1% to about 2%, by weight of the composition.

Non-limiting examples of suitable wax particles include polyethylenes, hydrocarbons, modified polyethylenes, oxidized polyethylenes, N,N′ Bisstearoylethylenediamine, polytetrafluoroethylene, polytetrafluoroethylene modified polyethylenes, partially saponified esters, polypropylenes, amides, synthetic hydrocarbons and aliphatic compounds synthesized using the Fischer-Tropsch process, hydrocarbon waxes, and mixtures thereof.

Preferred wax particles include polyethylenes, modified polyethylenes, oxidized polyethylenes, polytetrafluoroethylene modified polyethylenes, polypropylenes, synthetic hydrocarbons and aliphatic compounds synthesized using the Fischer-Tropsch process, hydrocarbon waxes, and mixtures thereof. Polypropylenes, such as ME59740, which is available from Michelman, Inc., are most preferred.

### Cosmetically Acceptable Medium

The present compositions comprise a cosmetically acceptable medium. The cosmetically acceptable medium is present in an amount from about 20% to about 95% by weight of the composition. The level and species of the medium are selected according to the compatibility with other components and other desired characteristic of the product. A cosmetically acceptable medium may be selected such that the compositions may be in the form of, for example, a pourable liquid (under ambient conditions), a gel, or other like mediums.

Cosmetically acceptable mediums useful in the present invention include water and water solutions of lower alkyl alcohols. Lower alkyl alcohols useful herein are monohydric alcohols having 1 to 6 carbons, more preferably ethanol and isopropanol.

The pH of the present composition, measured neat, is preferably from about 3 to about 9, more preferably from about 4 to about 8. Buffers and other pH-adjusting agents can be included to achieve the desirable pH.

### Load-Sensitive Deposit

In one embodiment, the present composition comprises wax particles which form a load-sensitive deposit upon dilution of the hair care composition with water. The load-sensitive deposit forms upon dilution of the hair care composition with water at a ratio of water to personal cleansing composition of at least about 1:1.

To deliver positive hair volume and body effects through the use of a deposit onto the hair, while still maintaining positive conditioning and combing performance, the deposit must perform differently under rest conditions than under combing conditions. Because the amount of force used to comb hair is magnitudes greater than the amount of force that hairs exert on other hairs when the hair is at rest, a deposit on the hair which provides a hair friction profile which decreases as force applied to the hair changes can provide improved hair volume, body, and fullness, while maintaining a good conditioning and combing performance.

### Additional Components

The present compositions may further comprise one or more optional components known for use in hair care or personal care products, provided that the optional components are physically and chemically compatible with the essential components described herein, or do not otherwise unduly impair product stability, aesthetics, or performance. Individual concentrations of such optional components may range from about 0.001% to about 10%.

Non-limiting examples of optional components for use in the composition include cationic polymers, additional particles, conditioning agents (e.g., silicones, hydrocarbon oils, fatty esters), anti-dandruff agents, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, chelants, proteins, salts, skin active agents, sunscreens, UV absorbers, and vitamins.

The present compositions may also contain pigment materials such as nitroso, monoazo, diazo, carotenoid, triphenyl methanes, triaryl methanes, xanthenes, quinolines, oxazines, azines, anthraquinones, indigoids, thionindigoids, quinacridones, phthalocyanines, botanicals, and natural colors including water soluble dye components.

### Cationic Polymers

The present compositions may also contain a cationic polymer to aid in conditioning performance. Suitable cationic polymers have a cationic charge density from about 0.5 meq/g to about 7.0 meq/g, preferably from about 0.7 meq/g to about 3.5 meq/g, more preferably from about 1.2 meq/g to about 2.5 meq/g, at the pH of intended use of the shampoo composition, which pH will generally range from about pH 3 to about pH 9, preferably from about pH 4 to about pH 8. The pH of the compositions is measured neat. The average molecular weight of such suitable cationic polymers is between about 10,000 and about 10 million, preferably between about 50,000 and about 5 million, more preferably between about 100,000 and about 3 million.

Such cationic polymers may be present in the composition from about 0.01% to about 3%, preferably from about 0.05% to about 2.0%, more preferably from about 0.1% to about 1.0%, by weight of the composition.

Suitable cationic polymers comprise cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability, or aesthetics. Non-limiting examples of such counterions include halides (e.g., chloride, fluoride, bromide, iodide), sulfate, and methylsulfate.

Non-limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (Polyquaternium-16); copolymers of 1-vinyl-2-pyrrolidone and dimethylaminoethyl methacrylate (Polyquaternium-11); cationic diallyl quaternary ammonium-containing polymers, including, for example, dimethyldiallylammonium chloride homopolymer, copolymers of acrylamide and dimethyldiallylammonium chloride (Polyquaternium 6 and Polyquaternium 7, respectively); amphoteric copolymers of acrylic acid including copolymers of acrylic acid and dimethyldiallylammonium chloride (Polyquaternium 22), terpolymers of acrylic acid with dimethyldiallylammonium chloride and acrylamide (Polyquaternium 39), and terpolymers of acrylic acid with methacrylamidopropyl trimethylammonium chloride and methylacrylate (Polyquaternium 47). Preferred cationic substituted monomers are the cationic substituted dialkylaminoalkyl acrylamides, dialkylaminoalkyl methacrylamides, and combinations thereof.

Other suitable cationic polymers include those which conform to the formula:

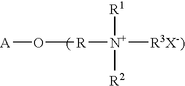

wherein A is an anhydroglucose residual group, such as a starch or cellulose anhydroglucose residual; R is an alkylene oxyalkylene, polyoxyalkylene, or hydroxyalkylene group, or combination thereof, R1, R2, and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) preferably being about 20 or less; and X is an anionic counterion as described in hereinbefore.

Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers, such as Polymer KG30M having an average charge density of 1.9 meq/g and a molecular weight of 1.5-2.0 million. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers include cationic guar gum derivatives, such as guar hydroxypropyltrimonium chloride, specific examples of which include the Jaguar series commercially available from Rhone-Poulenc Incorporated and the N-Hance series commercially available from Aqualon Division of Hercules, Inc. Cationic starches, such as waxy corn, and cationic cassia are also suitable cationic polymers.

When used, the cationic polymers herein are either soluble in the composition or are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

### Mono or Divalent Salt

The present personal care compositions may further comprise a mono or divalent salt. Typically addition of salts to a colloidal particle system results in decreased stability because the salt ions promote increased particle interaction. But when combined with neutral particles, such as the wax particles herein, salts are surprisingly useful to enhance stability of the composition. The salt contributes additional counter ions to the formulation which helps to prevent the complexing of the anionic surfactant and the optional cationic polymer in the formula. The salt also aids by preventing small amounts of the polymer/surfactant complexes from forming, reducing the overall clarity in the formula. Surfactant salts themselves are not included in the present salt definition but other salts are. Suitable salts comprise chlorides, phosphates, sulfates, nitrates, citrates, and halides. The counter ions of such salts comprise sodium, potassium, ammonium, magnesium, zinc, or other mono and divalent cations. Preferred salts comprise sodium chloride, ammonium chloride, sodium citrate, potassium chloride, magnesium chloride, and magnesium sulfate. Sodium chloride is most preferred. It is also recognized that these salts may serve as thickening aids or buffering aids in addition to their role as a complex inhibitor. The amount of salt varies with the type of surfactant and polymer, but is preferably present at a level of from about 0.01% to about 5%, more preferably from about 0.05% to about 3.5%, and still more preferably from about 0.1% to about 2% by weight of the personal care composition.

### Conditioning Agents

Conditioning agents include any material which is used to give a particular conditioning benefit to hair and/or skin. In hair treatment compositions, suitable conditioning agents are those which deliver one or more benefits relating to shine, softness, combability, antistatic properties, wet-handling, damage, manageability, body, and greasiness. The conditioning agents useful in the present compositions typically comprise a water-insoluble, water-dispersible, non-volatile, liquid that forms emulsified, liquid particles. Suitable conditioning agents are those characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics, or performance.

The concentration of the conditioning agent in the composition should be sufficient to provide the desired conditioning benefits, and as will be apparent to one of ordinary skill in the art. Such concentration can vary with the conditioning agent, the conditioning performance desired, the average size of the conditioning agent particles, the type and concentration of other components, and other like factors.

### Silicone Conditioning Agents

The conditioning agents useful in the present compositions are preferably water-insoluble silicone conditioning agents. The silicone conditioning agents may comprise volatile silicone, non-volatile silicone, or combinations thereof. Preferred are non-volatile silicone conditioning agents. The silicone conditioning agent particles may comprise a silicone fluid and may also comprise other ingredients, such as a silicone resin to improve silicone fluid deposition efficiency or enhance glossiness of the hair.

The silicone conditioning agent may be present from about 0.01% to about 10%, preferably from about 0.1% to about 5%, more preferably from about 0.2% to about 3%, by weight of the composition. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents preferably have a viscosity, as measured at 25° C., from about 20 to about 2,000,000 centistokes (“csk”), more preferably from about 1,000 to about 1,800,000 csk, even more preferably from about 50,000 to about 1,500,000 csk, more preferably from about 100,000 to about 1,500,000 csk.

Suitable non-volatile silicone oils may be selected from organo-modified silicones and fluoro-modified silicones. In one embodiment, the non-volatile silicone oil is an organo-modified silicone which comprises an organo group selected from the group consisting of alkyl groups, alkenyl groups, hydroxyl groups, amine groups, quaternary groups, carboxyl groups, fatty acid groups, ether groups, ester groups, mercapto groups, sulfate groups, sulfonate groups, phosphate groups, propylene oxide groups, and ethylene oxide groups.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

### Organic Conditioning Oils

The present compositions may also comprise at least one organic conditioning oil as the conditioning agent, either alone or in combination with other conditioning agents, such as the silicones described above. Such organic conditioning oils are present from about 0.05% to about 3%, preferably from about 0.08% to about 1.5%, more preferably from about 0.1% to about 1%, by weight of the composition.

### Hydrocarbon Oils

Suitable organic conditioning oils for use as conditioning agents in the present compositions include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₋₉. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used. Hydrocarbon polymers such as polybutene and polydecene are also useful. A preferred hydrocarbon polymer is polybutene, such as the copolymer of isobutylene and butene. A commercially available material of this type is L-14 polybutene from Amoco Chemical Corporation.

### Polyolefins

Organic conditioning oils for use in the present compositions can also include liquid polyolefins, more preferably liquid poly-α-olefins, more preferably hydrogenated liquid poly-α-olefins. Polyolefins for use herein are prepared by polymerization of C₄ to about C₁₄ olefinic monomers, preferably from about C₆ to about C₁₂.

Non-limiting examples of olefinic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents.

### Fatty Esters

Other suitable organic conditioning oils include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

Specific examples of preferred fatty esters include, but are not limited to, iso-propyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other suitable fatty esters are those known as polyhydric alcohol esters. Such polyhydric alcohol esters include alkylene glycol esters.

Still other suitable fatty esters are glycerides, including, but not limited to, mono-, di-, and tri-glycerides, preferably di- and tri-glycerides, more preferably triglycerides. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin, and soybean oil. Synthetic oils include, but are not limited to, triolein and tristearin glyceryl dilaurate.

### Other Conditioning Agents

### Quaternary Ammonium Compounds

Suitable quaternary ammonium compounds for use as conditioning agents in the present compositions comprise hydrophilic quaternary ammonium compounds with a long chain substituent having a carbonyl moiety, like an amide moiety, or a phosphate ester moiety or a similar hydrophilic moiety.

Examples of useful hydrophilic quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

Examples of other useful quaternary ammonium surfactants include, but are not limited to, Quaternium-33, Quaternium-43, isostearamidopropyl ethyldimonium ethosulfate, Quaternium-22 and Quaternium-26, or combinations thereof, as designated in the CTFA Dictionary.

Other hydrophilic quaternary ammonium compounds useful in the present compositions comprise Quaternium-16, Quaternium-27, Quaternium-30, Quaternium-52, Quaternium-53, Quaternium-56, Quaternium-60, Quaternium-61, Quaternium-62, Quaternium-63, Quaternium-71, and combinations thereof.

### Polyalkylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M, and mixtures thereof.

### Anti-Dandruff Actives

The present compositions may also comprise anti-dandruff actives. Suitable non-limiting examples of anti-dandruff actives include azoles, keratolytic agents, piroctone olamines (Octopirox®), ciclopirox and mixtures thereof. Such anti-dandruff actives should be physically and chemically compatible with the essential components of the composition, and should not otherwise unduly impair product stability, aesthetics, or performance.

Azole anti-microbials include imidazoles such as climbazole and ketoconazole.

The present invention may further comprise one or more keratolytic agents such as salicylic acid.

Additional anti-microbial actives may include extracts of melaleuca (tea tree) and charcoal.

When present in the composition, the anti-dandruff active is included in an amount from about 0.01% to about 5%, preferably from about 0.1% to about 3%, and more preferably from about 0.3% to about 2%, by weight of the composition.

### Suspending Agents

The present compositions may further comprise a suspending agent at concentrations effective for suspending water-insoluble material in dispersed form in the compositions or for modifying the viscosity of the composition. Such concentrations generally range from about 0.1% to about 10%, preferably from about 0.3% to about 5.0%, by weight of the composition, of suspending agent.

Suspending agents useful herein include anionic polymers and nonionic polymers. Useful herein are vinyl polymers such as cross linked acrylic acid polymers with the CTFA name Carbomer, a derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cyclonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

### Paraffinic Hydrocarbons

The present compositions may comprise one or more paraffinic hydrocarbons. Suitable paraffinic hydrocarbons comprise those materials which are known for use in hair care or other personal care compositions, such as those having a vapor pressure at 1 atm of equal to or greater than about 21° C. (about 70° F.). Non-limiting examples include pentane and isopentane.

### Propellants

The present compositions also may contain one or more propellants. Suitable propellants comprise those materials which are known for use in hair care or other personal care compositions, such as liquefied gas propellants and compressed gas propellants. Suitable propellants have a vapor pressure at 1 atm of less than about 21° C. (about 70° F.). Non-limiting examples of suitable propellants are alkanes, isoalkanes, haloalkanes, dimethyl ether, nitrogen, nitrous oxide, carbon dioxide, and mixtures thereof.

### Other Optional Components

The present compositions may comprise a fragrance.

The present compositions may also comprise vitamins such as vitamins B1, B2, B6, B12, C, pantothenic acid, pantothenyl ethyl ether, panthenol, biotin, and their derivatives, and vitamins A, D, E, and their derivatives. The compositions of the present invention may also contain amino acids such as asparagine, alanine, indole, glutamic acid, and their salts, and tyrosine, tryptamine, lysine, histadine, and their salts.

The present compositions may also comprise chelating agents.

### Method of Making

The present compositions may generally be made by mixing the ingredients together at either room temperature or at elevated temperature, e.g., about 72° C. Heat only needs to be used if solid dissoluble ingredients are in the composition. The ingredients are mixed at the batch processing temperature. Additional ingredients, including electrolytes, polymers, and particles, may be added to the product at room temperature.

### Method of Use

The personal care compositions herein are used in a conventional manner for providing both increased volume and superior styling and conditioning to hair. An effective amount of the composition for providing both increased volume and superior styling and conditioning to hair is applied to the hair, which has preferably been wetted with water, and then rinsed off. Such effective amounts generally range from about 1 g to about 50 g, preferably from about 1 g to about 20 g. Application to the hair typically includes working the composition through the hair such that most or all of the hair is contacted with the composition.

This method for providing both increased volume and superior styling and conditioning to hair comprises the steps of: (a) wetting the hair with water; (b) applying an effective amount of the personal care composition to the hair; and (c) rinsing the applied areas of hair with water. These steps can be repeated as many times as desired to achieve the desired cleansing and conditioning benefit.

The personal care compositions herein may be used as liquids, solids, semi-solids, gels, placed in a pressurized container with a propellant added, or used in a pump spray form. The viscosity of the product may be selected to accommodate the form desired.

### NON-LIMITING EXAMPLES

The compositions illustrated in the following Examples illustrate specific embodiments of the compositions of the present invention, but are not intended to be limiting thereof. Other modifications can be undertaken by the skilled artisan without departing from the spirit and scope of this invention. These exemplified embodiments of the composition of the present invention provide cleansing of hair and volumizing benefits with good wet conditioning and combing performance.

The compositions illustrated in the following Examples are prepared by conventional formulation and mixing methods, an example of which is set forth herein below. All exemplified amounts are listed as weight percents and exclude minor materials such as diluents, preservatives, color solutions, imagery ingredients, botanicals, and so forth, unless otherwise specified.

The present compositions may be prepared using conventional formulation and mixing techniques. Where melting or dissolution of solid surfactants or wax components is required these can be added to a premix of the surfactants, or some portion of the surfactants, mixed and heated to melt the solid components, e.g., about 72° C. This mixture can then optionally be processed through a high shear mill and cooled, and then the remaining components are mixed in. The compositions of the present invention, prior to the addition of materials such as gellants or propellants, typically have a viscosity from about 2,000 cps to about 20,000 cps. The viscosity of the composition can be adjusted by conventional techniques including addition of sodium chloride or ammonium xylenesulfonate as needed. The listed formulations, therefore, comprise the listed components and any minor materials associated with such components.

The following are representative of shampoo compositions of the invention:

| | EXAMPLE | | | | | | | |
|---|---|---|---|---|---|---|---|---|
| COMPOSITION | 1 | 2 | 3 | 4 | 5 | 6 | 7 | 8 |
| Sodium Laureth-3 Sulfate | 10 | 14 | 14 | 14 | 10 | 10 | 10 | 10 |
| Sodium Lauryl Sulfate | 6 | 2 | 2 | 2 | 6 | 6 | 6 | 6 |
| Ammonium Laureth-3 Sulfate | | | | | | | | |
| Ammonium Lauryl Sulfate | | | | | | | | |
| Cocamidopropylbetaine | | | | | | | | |
| Polyquat 10 (1) | 0.25 | | | | 0.15 | 0.25 | 0.5 | |
| Polyquat 10 (2) | | 0.25 | | | | | | |
| Polyquat 10 (3) | | | 0.25 | | | | | |
| Polyquat 10 (4) | | | | 0.5 | | | | 0.25 |
| Wax Microemulsion (5) | 1.0 | 1.0 | 2.0 | 2.0 | 1.5 | 2.0 | 0.5 | 4.0 |
| Dimethicone | | | | | 1 | 0.5 | | |
| microemulsion | | | | | | | | |
| Salicylic Acid | 0.5 | 1.0 | 0.5 | 2.0 | | | | |
| Cocamide MEA | 0.8 | 0.8 | 0.8 | 0.8 | 0.8 | 0.8 | 0.8 | 0 |
| Perfume Solution | 0.7 | 0.7 | 0.7 | 0.7 | 0.7 | 0.7 | 0.7 | 0.7 |
| Citric Acid | 0.23 | 0.23 | 0.23 | 0.23 | 0.23 | 0.23 | 0.23 | 0.23 |
| Sodium Benzoate | 0.25 | 0.25 | 0.25 | 0.25 | 0.25 | 0.25 | 0.25 | 0.25 |
| Sodium Chloride | 1.5 | 2.0 | 1.0 | 2.0 | 1.5 | 1.5 | 1.5 | 1.5 |
| Water and Minors (QS to 100%) | | | | | | | | |

(1) Polymer KG30M available from Amerchol
(2) Polymer JP available from Amerchol
(3) Polymer KG-4M available from Amerchol
(4) Polymer JR-30M available form Amerchol
(5) Wax microemulsion ME59740 available from Michelman particle size of 0.024 um, melt temperature of 142-148 C.

| | EXAMPLE | | | | | | | | | |
|---|---|---|---|---|---|---|---|---|---|---|
| COMPOSITION | 9 | 10 | 11 | 12 | 13 | 14 | 15 | 16 | 17 | 18 |
| Sodium Laureth-3 Sulfate | 10 | 10 | 10 | 8 | 4 | | | 10 | 10 | |
| Sodium Lauryl Sulfate | 6 | 6 | 6 | 8 | 8 | | | 6 | 6 | |
| Ammonium Laureth-3 Sulfate | | | | | | 10 | 12.5 | | | 8 |
| Ammonium Lauryl Sulfate | | | | | | 6 | 1.5 | | | 8 |
| Cocamidopropylbetaine | | | | | 4 | | 2.7 | | | |
| Polyquat 10 (1) | 0.25 | 0.25 | 0.25 | | 0.5 | 0.25 | 0.5 | 0.25 | | |
| Polyquat 10 (2) | | | | 0.25 | | | | | 0.25 | |
| Polyquat 10 (3) | | | | | | | | | | 0.75 |
| Polyquat 10 (4) | | | | | | | | | | |
| Wax Microemulsion (5) | 1.0 | 5.0 | 1.0 | 2.0 | 2.0 | 1.0 | 2.0 | 1.5 | 1.0 | 1.0 |
| Dimethicone microemulsion | | | | | | | 0.25 | | | |
| Climbazole | | | | | | | | 1.0 | | |
| Salicylic Acid | | | | | | | 0.5 | | | 1.0 |
| Cocamide MEA | 0.8 | 0.8 | 0.8 | 0.8 | 0 | 0.8 | 0.8 | 0.8 | 0.8 | 0 |
| Perfume Solution | 0.7 | 0.7 | 0.7 | 0.7 | 0.7 | 0.55 | 0.55 | 0.7 | 0.7 | 0.55 |
| Citric Acid | 0.23 | 0.23 | 0.23 | 0.23 | 0.23 | 0.04 | 0.04 | 0.2 | 0.23 | 0.04 |
| Sodium Benzoate | 0.25 | 0.25 | 0.25 | 0.25 | 0.25 | 0.25 | 0.25 | 0.25 | 0.25 | 0.25 |
| Sodium Chloride | 1.5 | 1.5 | 1.5 | 0.2 | 0.5 | 0.5 | 3.0 | 2.0 | 0.0 | 1.0 |
| Water and Minors (QS to 100%) | | | | | | | | | | |

(1) Polymer KG30M available from Amerchol
(2) Polymer JP available from Amerchol
(3) Polymer KG-4M available from Amerchol
(4) Polymer JR-30M available form Amerchol
(5) Wax microemulsion ME59740 available from Michelman

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A personal care composition comprising:
a) from about 5 wt. % to about 50 wt. % of a detersive surfactant,
b) from about 0.05 wt. % to about 20 wt. % of at least one wax particle having a melting point of at least about 100° C. and an average mean particle size as measured in said personal care composition less than about 0.15 μm,
c) at least about 20 wt. % of a cosmetically acceptable medium; and
wherein said personal care composition is substantially clear.

2. A personal care composition according to claim 1, wherein said composition forms a load sensitive deposit.

3. A personal care composition according to claim 1, wherein said at least one wax particle is selected from the group consisting of polyethylenes, hydrocarbons, modified polyethylenes, oxidized polyethylenes, N,N′ Bisstearoylethylenediamine, polytetrafluoroethylene, polytetrafluoroethylene modified polyethylenes, partially saponified esters, polypropylenes, amides, synthetic hydrocarbons and aliphatic compounds synthesized using the Fischer-Tropsch process, hydrocarbon waxes, and mixtures thereof.

4. A personal care composition according to claim 3, wherein said at least one wax particle is polypropylene wax.

5. A personal care composition according to claim 1, further comprising a cationic polymer.

6. A personal care composition according to claim 5, wherein said cationic polymer has a charge density from about 0.5 meq/g to about 7 meq/g and a molecular weight of from about 10,000 to about 10,000,000.

7. A personal care composition according to claim 6, wherein said cationic polymer has a charge density from about 0.7 meq/g to about 3.5 meq/g.

8. A personal care composition according to claim 6, wherein said cationic polymer has a charge density from about 1.2 meq/g to about 2.5 meq/g.

9. A personal care composition according to claim 5, wherein said cationic polymer is selected from the group consisting of cationic cellulose derivatives, cationic guar gum derivatives, cationic starches, and cationic cassia.

10. A personal care composition according to claim 1, further comprising a conditioning agent.

11. A personal care composition according to claim 10, wherein said conditioning agent is selected from the group consisting of silicone conditioning agents, hydrocarbon oils, polyolefins, fatty esters, and mixtures thereof.

12. A personal care composition according to claim 1, further comprising one or more additional components selected from the group consisting of anti-dandruff agents, and conditioning agents.

13. A personal care composition according to claim 12, wherein said anti-dandruff agents are selected from the group consisting of climbazole and salicylic acid.

14. A personal care composition according to claim 1, wherein said wax particle has a melting point of from about 105° C. to about 200° C.

15. A personal care composition comprising:
a) from about 5 wt. % to about 50 wt. % of a detersive surfactant,
b) from about 0.05 wt. % to about 20 wt. % of at least one wax particle having a melting point of at least about 100° C. and an average mean particle size as measured in said personal care composition of less than about 0.15 μm,
c) from about 0.01 wt. % to about 5 wt. % of a mono or divalent salt,
d) at least about 20 wt. % of a cosmetically acceptable medium; and
wherein said personal care composition is substantially clear.
